(19) United States
(12) Patent Application Publication
(10) Pub. No.: US 2021/0007674 A1
(43) Pub. Date: Jan. 14, 2021

(54) METHOD OF ESTIMATION OF THE QUALITY OF A HEART RATE SIGNAL

(71) Applicants: Commissariat à I'Energie Alomique et aux Energies Alternatives, Parise (FR); UNIVERSITE GRENOBLE ALPES, Saint Martin D'Heres (FR)

(72) Inventors: Gaël VILA, Grenoble (FR); Christelle GODIN, Grenoble (FR); Aurélie CAMPAGNE, Fontaine (FR); Sylvie CHARBONNIER, Echirolles (FR)

(21) Appl. No.: 16/946,805

(22) Filed: Jul. 7, 2020

(30) Foreign Application Priority Data
Jul. 10, 2019 (FR) 1907758

(51) Int. Cl. A61B 5/00; A61B 5/024; G16H 40/67; G16H 50/20

(52) U.S. Cl. CPC A61B 5/7221; A61B 5/024; A61B 5/0245; G16H 50/20; A61B 5/02433; G16H 40/67

(57) ABSTRACT

The present disclosure concerns a method of estimation, by means of an electronic processing device (104), of the quality of a heart rate signal delivered by a heart rate sensor during an acquisition phase of duration W, said signal including a sequence of N samples IBIᵢ each having a value representative of a duration between two successive heartbeats detected by the sensor, N being an integer greater than or equal to 2 and i being an integer in the range from 1 to N, the method including a step of calculating, by means of the electronic processing device, of a default indicator L representative of the difference between duration W of the acquisition phase and the sum of the values of the samples IBIᵢ of the signal.

### FIELD

The present disclosure generally concerns the field of systems using heart rate sensors, and more particularly aims at a method of estimation of the quality of the heart rate signals delivered by such sensors.

### BACKGROUND

A subject's heart rate is a physiological parameter used in many applications, for example stress control applications, applications of control and/or of prevention of certain chronic diseases, or also in applications of control of the subject's physical activity.

In certain applications, the subject has to continuously carry a heart rate sensor for long periods, for example, throughout the day and/or the night, in ambulatory conditions. Many sensors adapted to such applications are now available for sale.

For certain applications, the quality of the signals supplied by existing sensors is however not always sufficient. This is particularly true for applications using the short-term variability of the heart rate, for example, stress control applications. In such applications, a few missed beats may be sufficient to significantly alter the analyses.

### SUMMARY

An embodiment provides a method of estimation, by means of an electronic processing device, of the quality of a heart rate signal delivered by a heart rate sensor during an acquisition phase of duration W, said signal comprising a sequence of N samples IBIᵢ each having a value representative of a duration between two successive heartbeats detected by the sensor, N being an integer greater than or equal to 2 and i being an integer in the range from 1 to N, the method comprising a step of calculation, by means of the electronic processing device, of a default indicator L representative of the difference between duration W of the acquisition phase and the sum of the values of the signal samples IBIᵢ.

According to an embodiment of the present invention, the default indicator L is representative of a percentage of heartbeats missed by the sensor during the acquisition phase.

According to an embodiment of the present invention, default indicator L is defined by the following formula:

$$L = \frac{W - {\sum }_{i = 1}^{N} {IBI}_{i}}{W}$$

According to an embodiment of the present invention, duration W of the acquisition phase is in the range from 20 to 120 seconds.

According to an embodiment of the present invention, the method further comprises a step of comparison of default indicator L with a predefined threshold TH and a step of decision, based on the result of the comparison, to take into account or not the heart rate signal.

According to an embodiment of the present invention, during the decision step, the heart rate signal is taken into account only if default indicator L is smaller than threshold TH.

According to an embodiment of the present invention, threshold TH is defined as follows:

$$TH = \frac{τ}{100} + \frac{{min}_{i ∈ 1 … N} {IBI}_{i}}{W}$$

where τ is a tolerance margin between 0 and 100 defining a maximum tolerated percentage of heartbeats missed by the sensor during the acquisition phase.

According to an embodiment of the present invention, margin τ is in the range from 0 to 20.

Another embodiment provides a system comprising a heart rate sensor and an electronic processing device, the electronic processing device being configured to implement a method of estimation of the quality of a heart rate signal delivered by the sensor such as defined hereabove.

### BRIEF DESCRIPTION OF THE DRAWINGS

The foregoing and other features and advantages will be discussed in detail in the following non-limiting description of specific embodiments in connection with the accompanying drawings, in which:

FIG. 1 schematically shows in the form of blocks an example of a system comprising a heart rate sensor and an electronic processing device capable of implementing a method of estimation of the quality of a heart rate signal according to an embodiment;

FIG. 2 is a diagram showing as an illustration an example of a heart rate signal; and

FIG. 3 schematically shows in the form of a blocks an example of a method of estimation of the quality of a heart rate signal according to an embodiment.

### DETAILED DESCRIPTION OF THE PRESENT EMBODIMENTS

Like features have been designated by like references in the various figures. In particular, the structural and/or functional features that are common among the various embodiments may have the same references and may dispose identical structural, dimensional and material properties.

For the sake of clarity, only the steps and elements that are useful for an understanding of the embodiments described herein have been illustrated and described in detail. In particular, the manufacturing of a heart rate sensor capable of delivering the heart rate signals processed by the quality estimation method of the present application has not been detailed, the described embodiments being compatible with all or most known heart rate sensors, or the manufacturing of such a sensor being within the abilities of those skilled in the art based on the indications of the present disclosure. Further, the manufacturing of an electronic processing device capable of implementing the method described hereafter of estimation of the quality of a heart rate signal has not been detailed, the manufacturing of such a device being within the abilities of those skilled in the art based on the indications of the present disclosure. Further, the applications capable of using the heart rate signals analyzed by the method of the present application have not been detailed, the described embodiments being compatible with all or most application capable of taking advantage of the provision of an indicator representative of the quality of a heart rate signal delivered by a heart rate sensor.

Unless specified otherwise, when reference is made to two elements connected together, this signifies a direct connection without any intermediate elements other than conductors, and when reference is made to two elements coupled together, this signifies that these two elements can be connected or they can be coupled via one or more other elements.

Unless specified otherwise, the expressions “around”, “approximately”, “substantially” and “in the order of” signify within 10%, and preferably within 5%.

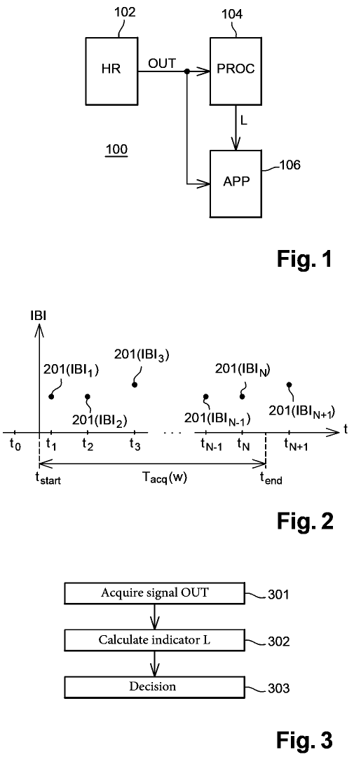

FIG. 1 schematically shows in the form of blocks an example of a system 100 comprising a heart rate sensor 102 (HR) and an electronic processing device 104 (PROC) configured to implement a method of estimation of the quality of a heart rate signal delivered by sensor 102.

Sensor 102 may be a sensor of electrocardiograph type, measuring the electric activity of the heart by means of electrodes placed in contact with the surface of the subject's skin. As a variant, sensor 102 may be a sensor of photoplethysmograph type, measuring the variations of a light signal for example, an infrared signal, having a blood vessel of the subject placed on its path (for example, at the level of a wrist of the user in the case of a bracelet-type sensor). More generally, the described embodiments apply to any type of sensor capable of measuring a signal representative of the subject's heartbeats.

A sensor delivering an output signal OUT in the form of a sequence of samples, each having a value representative of a period between two successive heartbeats detected by the sensor, is more particularly considered herein. It can then be spoken of an instantaneous heart rate signal since each sample is representative of the current interbeat interval and thus of the current (instantaneous) heart rate of the subject.

In the example of FIG. 1, signal OUT is transmitted to an electronic application device 106 (APP) configured to implement a method using signal OUT, for example, a stress control method, a method of control and/or of prevention of certain chronic diseases, or also a method of control of the subject's physical activity. The link between sensor 102 and device 106 is for example a wire link.

To generate signal OUT, sensor 102 comprises an internal processing circuit, not detailed in the drawing. The internal processing circuit generates signal OUT from a raw analog signal delivered by an acquisition element (not detailed) of the sensor. In practice, according to the type of sensor used and according to the conditions of use of the sensor, for example, in case of a mispositioning of the sensor, certain portions of the analog signal supplied by the acquisition element of the sensor may be too noisy to perform a reliable heartbeat detection. The portions can be identified by the internal processing device of the sensor and are then not taken into account to generate signal OUT. Further, certain calculated values of interval between beats may be deemed aberrant by the internal processing device of the sensor, for example, if they do not comply with a predetermined relevancy criterion, and are thus not transmitted in the output signal OUT Of the sensor. Thus, certain heartbeats of the subject are not taken into account to generate signal OUT. In other words, the signal OUT supplied by sensor 102 is a signal rid or cleaned of possible outliers. The method implemented by sensor 102 to generate, from the noise signal, an output signal OUT rid of outliers, will not be detailed, the described embodiments being compatible with all or most known methods for cleaning a raw signal delivered by a heart rate sensor.

According to the type of application implemented by device 106, the missing beats may result in significantly altering the obtained results. In particular, beat defaults are particularly problematic for applications using the short-term variability of the heart rate, for example, stress control applications.

For this reason, in the system of FIG. 1, the output signal OUT of sensor 102 is further transmitted to device 104, which implements a method of estimation of the quality of signal OUT. More particularly, device 104 calculates an indicator L of the quality of signal OUT. Indicator L is transmitted to application device 106 which, based on this indicator, determines whether signal OUT may or not be used by the application. The link between sensor 102 and processing device 104 is for example a wire link. The link between processing device 104 and application device 106 may also be a wire link.

Processing device 104 may comprise a microprocessor or any other processing circuit capable of implementing the method of calculation of indicator L described hereafter. As an example, application device 106 and processing circuit 104 comprise common elements, for example, a same microprocessor.

FIG. 2 is a diagram showing as an illustration an example of a heart rate signal. More particularly, the diagram of FIG. 2 shows the variation over time t (in abscissas), of the subject's interbeat interval IBI (in ordinates).

In FIG. 2, times t₀, t₁, t₂, t₃, . . . t_{N-1}, t_{N}, t_{N+1} have been shown on the axis of abscissas, respectively corresponding to times of occurrence of successive heartbeats of the subject. For each of times tᵢ, i being an integer in the range from 1 to N+1, FIG. 2 shows a point 201 having time tᵢ as an abscissa and a value IBIᵢ=tᵢ−tᵢ₋₁ corresponding to the time interval elapsed between the heartbeats of times and ti as an ordinate.

The output signal OUT of sensor 102 is for example formed by the sequence of values IBI₁, IBI₂, IBI₃, . . . , IBI_{N-1}, IBI_{N}, IBI_{N+1}.

FIG. 3 schematically shows in the form of blocks an example of a method of estimation of the quality of the heart rate signal OUT delivered by sensor 102, implemented by processing device 104.

The method of FIG. 3 comprises a step 301 of acquisition of signal OUT during an acquisition phase T_{acq} of duration W, from a time tₛₜₐᵣₜ to a time t_{end}. Duration W of acquisition phase T_{acq} is for example in the range from 20 to 120 seconds. The described embodiments are however not limited to this specific case.

The signal OUT acquired during acquisition phase T_{acq} is formed of N successive samples IBI₁, . . . , IBI_{N}, each representative of a duration between two successive heartbeats of the subject.

When all the subject's heartbeats are effectively taken into account within sensor 102 to generate signal OUT, the sum of the values of the N samples IBI₁, . . . , IBI_{N} acquired during acquisition phase T_{acq} is close to duration W of acquisition phase T_{acq}. More particularly, considering that the time tₛₜₐᵣₜ of beginning of acquisition phase T_{acq} may be between two successive heartbeats of the subject, between beats to and ti in the shown example, and that the time t_{end} of end of acquisition phase T_{acq} may be between two successive heartbeats of the subject, between beats t_{N} and t_{N+1} in the shown example, the following relation is respected:

$${\sum }_{i = 1}^{N} {IBI}_{i} - {IBI}_{1} < W < {\sum }_{i = 1}^{N} {IBI}_{i} + {IBI}_{N + 1} \qquad [ Eq . 1 ]$$

If however certain interbeat intervals of the patient have not been taken into account by sensor 102 and have accordingly not been transmitted in signal OUT, the sum of the values of the samples IBI₁, . . . , IBI_{N} acquired during acquisition phase T_{acq} may be substantially smaller than duration W of acquisition phase T_{acq}.

According to an aspect of the described embodiments, it is provided, during a step 302 subsequent to step 301, to calculate an indicator L representative of the difference between duration W of the acquisition phase and the sum of the values of the samples of the signal. This indicator, also called default indicator, is representative of the proportion of interbeat periods which have not been taken into account by sensor 102, and is used as an indicator of the quality of the output signal OUT of sensor 102.

Indicator L is for example defined as follows:

$$L = \frac{W - {\sum }_{i = 1}^{N} {IBI}_{i}}{W} \qquad [ Eq . 2 ]$$

Indicator L is thus representative of the percentage of beats which have not been taken into account in acquisition window T_{acq}.

Another way to express indicator L is to consider a theoretical number Nₜₕ of samples which should have been acquired during acquisition phase T_{acq}, defined as follows:

$${N}_{th} = \frac{W}{μ} \qquad [ Eq . 3 ]$$

where μ designates the average of the values of samples IBI₁, . . . , IBI_{N}, that is:

$${N}_{th} = \frac{W * N}{{\sum }_{i = 1}^{N} {IBI}_{i}} \qquad [ Eq . 4 ]$$

One then has:

$$L = \frac{{N}_{th} - N}{{N}_{th}} \qquad [ Eq . 5 ]$$

FIG. 3 further shows a step 303, subsequent to step 302, of decision, based on indicator L, of taking into account or not the signal OUT acquired during acquisition phase T_{acq}. Decision step 303 may be implemented by application device 106 itself, or by processing device 104. In this last case, only a binary value representative of the result of the decision may be transmitted to application device 106.

The decision step for example comprise comparing indicator L with a predefined threshold TH, and taking into account the signal OUT acquired during acquisition phase T_{acq} only if indicator L is smaller than threshold TH.

Threshold TH is for example defined as follows:

$$TH = \frac{τ}{100} + \frac{{min}_{i ∈ 1 … N} {IBI}_{i}}{W} \qquad [ Eq . 6 ]$$

As an example, the acquisition time window T_{acq} of duration W is a sliding window, the method of FIG. 3 being repeated each time a new sample of signal OUT is supplied by sensor 102. This enables to detect and to take into account all the portions of the signal OUT of duration W complying with the quality criterion defined at step 303.

Various embodiments and variants have been described. Those skilled in the art will understand that certain features of these various embodiments and variants may be combined and other variants will occur to those skilled in the art. In particular, the described embodiments are not limited to the above-described examples of numerical parameters.

Finally, the practical implementation of the embodiments and variants described herein is within the capabilities of those skilled in the art based on the functional indications provided hereinabove.

Such alterations, modifications, and improvements are intended to be part of this disclosure, and are intended to be within the spirit and the scope of the present invention. Accordingly, the foregoing description is by way of example only and is not intended to be limiting. The present invention is limited only as defined in the following claims and the equivalents thereto.

1. A method of estimation, by means of an electronic processing device, of the quality of a heart rate delivered by a heart rate sensor during an acquisition phase of duration W, said signal being delivered rid of possible outliers and comprising a sequence of N samples IBIᵢ each having a value representative of a duration between two successive heartbeats detected by the sensor, N being an integer greater than or equal to 2 and i being an integer in the range from 1 to N, the method comprising a step of calculation, by means of the electronic processing device of a ratio or a difference between duration W of the acquisition phase and the sum of the values of the samples IBIᵢ of the signal and a step of delivery of a default indicator L which is a function of this ratio or of this difference.

2. The method according to claim 1, wherein the default indicator L is representative of a percentage of heartbeats missed by the sensor during the acquisition phase.

3. The method of claim 1, wherein the default indicator L is defined by the following formula:
$$L = \frac{W - {\sum }_{i = 1}^{N} {IBI}_{i}}{W}$$

4. The method of claim 1, wherein the duration W of the acquisition phase is in the range from 20 to 120 seconds.

5. The method of claim 1, further comprising a step of comparison of default indicator L with a predefined threshold TH and a step of decision, based on the result of the comparison, to take into account or not the heart rate signal.

6. The method according to claim 5, wherein, during the decision step, the heart rate signal is taken into account only if default indicator L is smaller than threshold TH.

7. The method according to claim 5, wherein threshold TH is defined as follows:
$$TH = \frac{τ}{100} + \frac{{min}_{i ∈ 1 … N} {IBI}_{i}}{W}$$
where τ is a tolerance margin between 0 and 100 defining a maximum tolerated percentage of heartbeats missed by the sensor during the acquisition phase.

8. The method according to claim 7, wherein margin τ is in the range from 0 to 20.

9. A system comprising a heart rate sensor and an electronic processing device, the electronic processing device being configured to implement a method of estimation of the quality of a heart rate signal delivered by the sensor according to claim 1.
